(19) United States
(12) Patent Application Publication
(10) Pub. No.: US 2013/0165654 A1
(43) Pub. Date: Jun. 27, 2013

(54) PROCESSES FOR PREPARING PEMETREXED

(71) Applicants: Rajasekhar Kadaboina, Hyderabad (IN); Sekhar Munaswamy Nariyam, Hyderabad (IN); Veerender Murki, Hyderabad (IN); Amarendhar Manda, Hyderabad (IN); Raghupati Rama Vinjamuri, Hyderabad (IN); Nageshwar Gunda, Hyderabad (IN)

(72) Inventors: Rajasekhar Kadaboina, Hyderabad (IN); Sekhar Munaswamy Nariyam, Hyderabad (IN); Veerender Murki, Hyderabad (IN); Amarendhar Manda, Hyderabad (IN); Raghupati Rama Vinjamuri, Hyderabad (IN); Nageshwar Gunda, Hyderabad (IN)

(73) Assignees: DR. REDDY'S LABORATORIES, INC., Bridgewater, NJ (US); DR. REDDY'S LABORATORIES LTD., Hyderabad (IN)

(21) Appl. No.: 13/371,546

(22) Filed: Feb. 13, 2012

Related U.S. Application Data
(63) Continuation of application No. PCT/US2010/045429, filed on Aug. 13, 2010.
(60) Provisional application No. 61/304,129, filed on Feb. 12, 2010.

(30) Foreign Application Priority Data
Aug. 13, 2009 (IN) 1932/CHE/2009

(51) Int. Cl. C07D 487/04; C07C 309/30; C07C 303/32

(52) U.S. Cl. CPC C07D 487/04; C07C 303/32; C07C 309/30
USPC 544/280

(57) ABSTRACT

The present invention relates to pemetrexed disodium substantially free from specific process-related impurities, and processes for the preparation thereof.

The present application relates to processes for preparing pemetrexed and its salts.

A chemical name for the drug compound “pemetrexed” is 2-[4-[2-(4-amino-2-oxo-3,5,7-triazabicyclo[4.3.0]nona-3,8,10-trien-9-yl)ethyl]benzoyl]amino-pentanedioic acid. The drug compound having the adopted name “pemetrexed disodium” is also known by the chemical name L-glutamic acid, N-[4-[2-(2-amino-4,7-dihydro-4-oxo-1H-pyrrolo[2,3-d]pyrimidin-5-yl)ethyl]benzoyl]-disodium salt, heptahydrate, and is represented by the structure of Formula I.

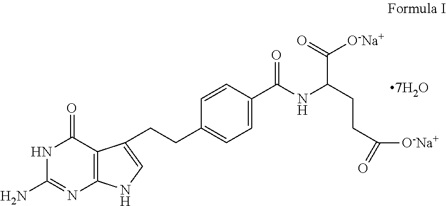

Pemetrexed is an anti-folate anti-neoplastic agent that exerts its action by disrupting folate-dependent metabolic processes essential for cell replication. It is believed to work by inhibiting three enzymes that are required in purine and pyrimidine biosynthesis-thymidylate synthase (TS), dihydrofolate reductase (DHFR), and glycinamide ribonucleotide formyl transferase (GARFT). Pemetrexed disodium heptahydrate is the active ingredient in a lyophilized powder for intravenous infusion, sold by Eli Lilly and Company as ALIMTA®.

Taylor et al., in U.S. Pat. No. 5,344,932, describe pemetrexed, its related compounds, and pharmaceutically acceptable cations.

U.S. Pat. No. 5,416,211 describes a process for the preparation of pemetrexed, as represented in Scheme 1.

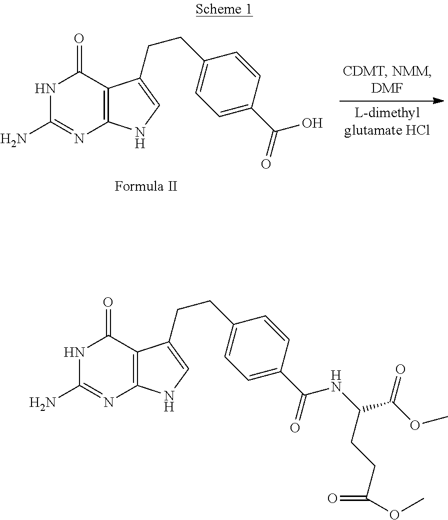

C. J. Barnett et al., “A Practical Synthesis of Multitargeted Antifolate LY231514,” Organic Process Research & Development, Volume 3 (3), pages 184-188, 1999 describes a process for the preparation of pemetrexed disodium. The process is represented in Scheme 2.

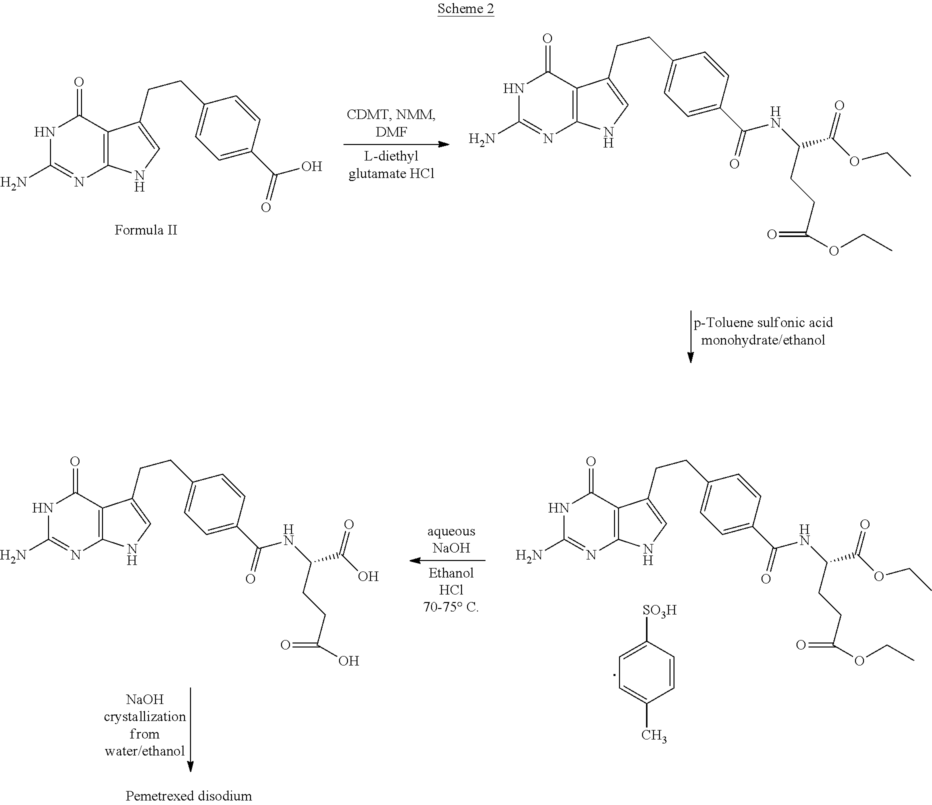

Chelius et al., in International Application Publication No. WO 01/14379 A2, disclose pemetrexed disodium crystalline hydrate Form I and processes for preparation thereof. Processes are represented in Schemes 3 and 4.

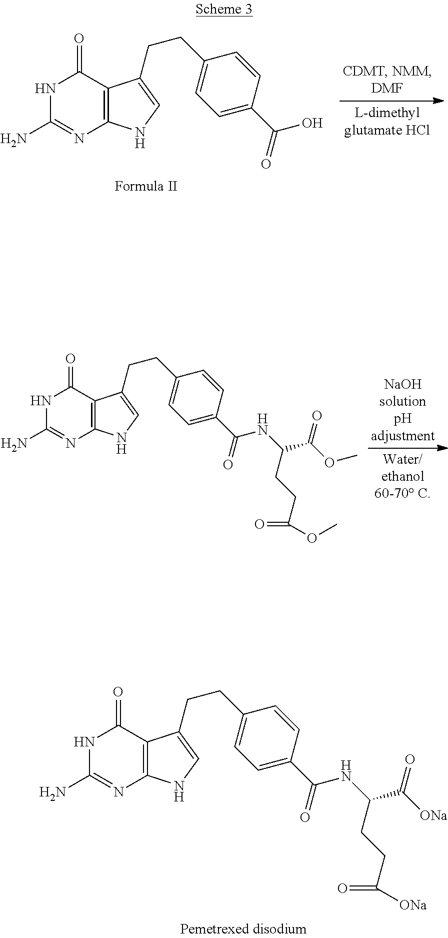

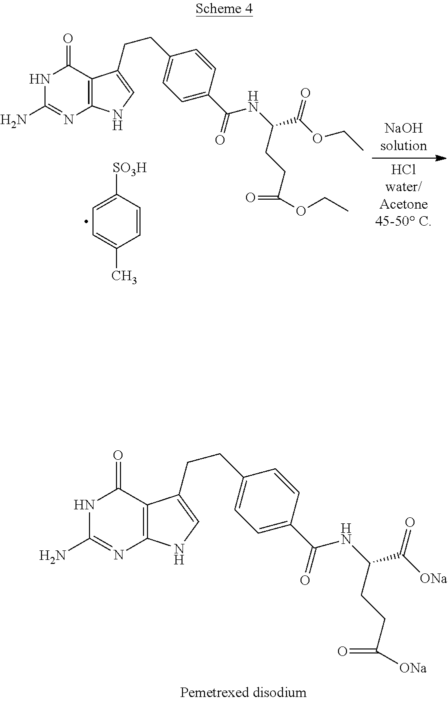

It has now been found that, when the condensation reaction is performed between 4-[2-(2-amino-4,7-dihydro-4-oxo-1H-pyrrolo[2,3-d]pyrimidin-5-yl)ethyl]benzoic acid of Formula II and L-dialkyl glutamate HCl, in the presence of an amide as a solvent, it results in the formation of an impurity of Formula A, which subsequently after de-esterification results in the formation of the impurity of Formula B or its salt (the structures of these compounds being shown herein below). The process using the amide solvent N,N-dimethylformamide is summarized in Scheme 5.

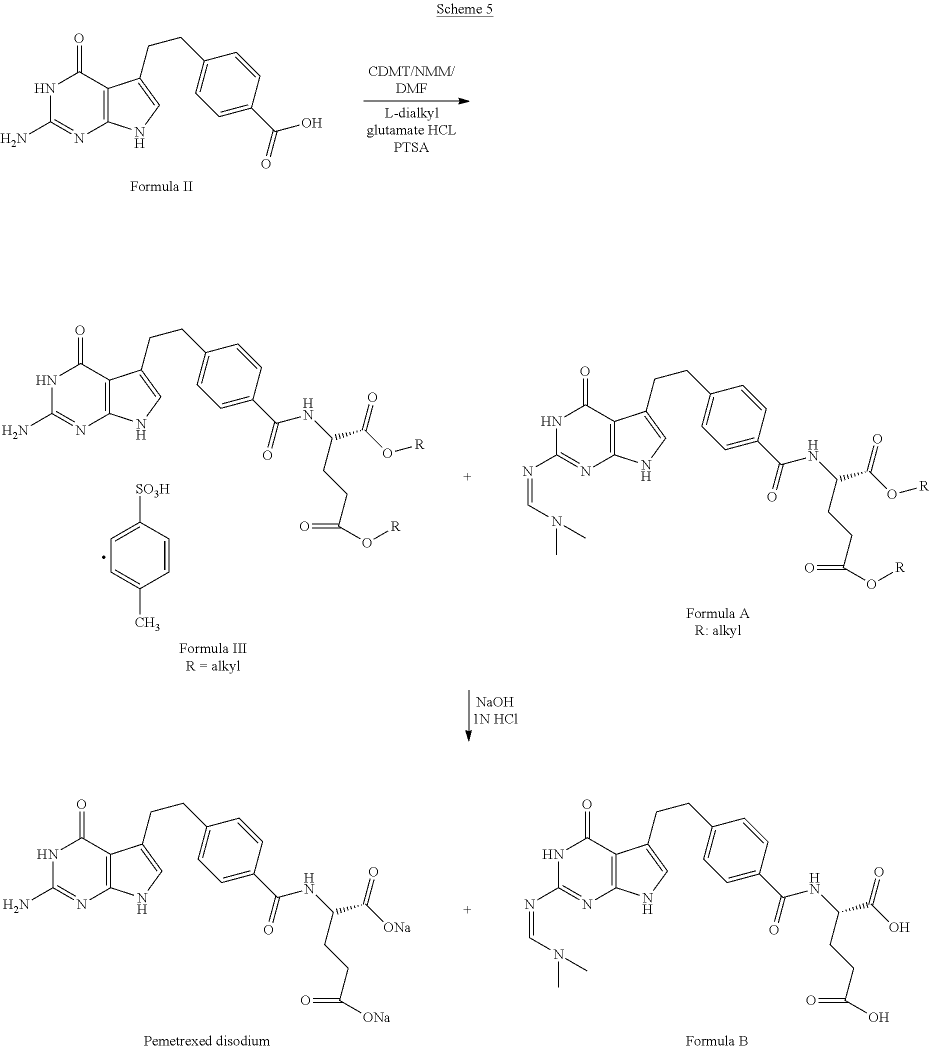

Further, it has now been observed that during de-esterification, that is, hydrolysis of a dialkyl ester of pemetrexed or its salt in an alkaline medium at temperatures about ambient or above (e.g., >30° C.), there is formation of a chiral impurity, which is represented by Formula C.

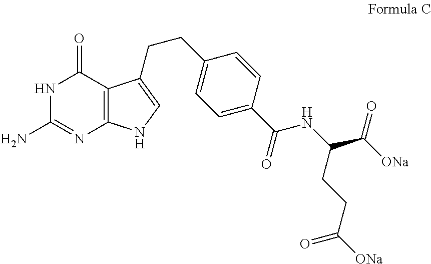

Impurities in any active pharmaceutical ingredient (API) are undesirable, and, in extreme cases, might even be harmful to a patient. Furthermore, the undesired impurities may reduce the availability of the API in the pharmaceutical composition and can decrease the stability of a pharmaceutical dosage form. Therefore, there is a need for new processes for the preparation of pemetrexed disodium, which are cost effective, industrially viable, and provide pemetrexed disodium substantially free of impurities.

### SUMMARY

In an aspect, the present application provides processes for preparing N-[4-[2-(2-amino-4,7-dihydro-4-oxo-1H-pyrrolo[2,3-d]pyrimidin-5-yl)ethyl]benzoyl]-L-glutamic acid dialkyl ester p-toluenesulfonate salt of formula,

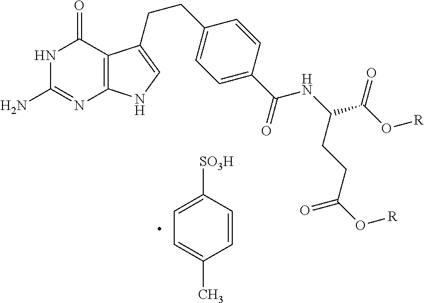

wherein R=alkyl
comprising:

a) reacting 4-[2-(2-amino-4,7-dihydro-4-oxo-1H-pyrrolo[2,3-d]pyrimidin-5-yl)ethyl] benzoic acid of Formula II

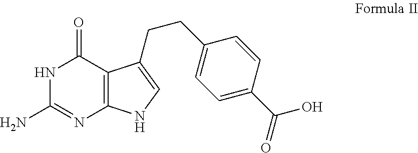 [SMILES: Nc1nc2[nH]cc(CCc3ccc(C(=O)O)cc3)c2c(=O)[nH]1]

with L-dialkyl glutamate HCl, in the presence of N-methylpyrrolidone (NMP), to obtain N-[4-[2-(2-amino-4,7-dihydro-4-oxo-1H-pyrrolo[2,3-d]pyrimidin-5-yl)ethyl]benzoyl]-L-glutamic acid dialkyl ester; and

b) reacting N-[4-[2-(2-amino-4,7-dihydro-4-oxo-1H-pyrrolo[2,3-d]pyrimidin-5-yl)ethyl]benzoyl]-L-glutamic acid dialkyl ester with p-toluenesulfonic acid, in an organic solvent.

In an specific aspect, the present application provides processes for preparing N-[4-[2-(2-amino-4,7-dihydro-4-oxo-1H-pyrrolo[2,3-d]pyrimidin-5-yl)ethyl]benzoyl]-L-glutamic acid dimethyl ester p-toluenesulfonate salt of Formula III,

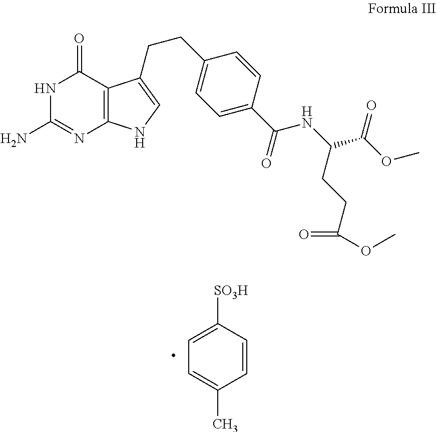

substantially free from its impurity of Formula A, embodiments comprising:

a) reacting 4-[2-(2-amino-4,7-dihydro-4-oxo-1H-pyrrolo[2,3-d]pyrimidin-5-yl)ethyl] benzoic acid of Formula II

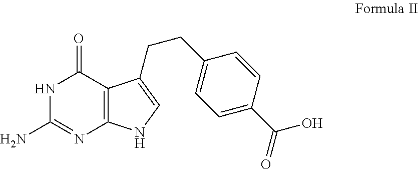 [SMILES: Nc1nc2[nH]cc(CCc3ccc(C(=O)O)cc3)c2c(=O)[nH]1]

with L-dimethyl glutamate HCl, in the presence of N-methylpyrrolidone (NMP), to obtain N-[4-[2-(2-amino-4,7-dihydro-4-oxo-1H-pyrrolo[2,3-d]pyrimidin-5-yl)ethyl]benzoyl]-L-glutamic acid dimethyl ester; and

b) reacting N-[4-[2-(2-amino-4,7-dihydro-4-oxo-1H-pyrrolo[2,3-d]pyrimidin-5-yl)ethyl]benzoyl]-L-glutamic acid dimethyl ester with p-toluenesulfonic acid, in an organic solvent.

In an aspect, the present application provides processes for the preparation of pemetrexed disodium, substantially free from impurities of Formulas A, B, and C, embodiments comprising:

i) reacting 4-[2-(2-amino-4,7-dihydro-4-oxo-1H-pyrrolo[2,3-d]pyrimidin-5-yl)ethyl] benzoic acid of Formula II:

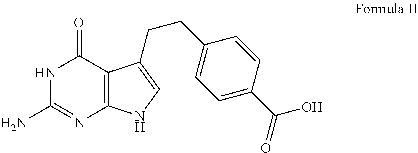 [SMILES: Nc1nc2[nH]cc(CCc3ccc(C(=O)O)cc3)c2c(=O)[nH]1]

with L-dimethyl glutamate hydrochloride, in the presence of N-methylpyrrolidone, to obtain N-[4-[2-(2-amino-4,7-dihydro-4-oxo-1H-pyrrolo[2,3-d]pyrimidin-5-yl) ethyl]benzoyl]-L-glutamic acid dimethyl ester;

ii) reacting N-[4-[2-(2-amino-4,7-dihydro-4-oxo-1H-pyrrolo[2,3-d]pyrimidin-5-yl)ethyl]benzoyl]-L-glutamic acid dimethyl ester with p-toluenesulfonic acid to provide N-[4-[2-(2-amino-4,7-dihydro-4-oxo-1H-pyrrolo[2,3-d]pyrimidin-5-yl)ethyl]benzoyl]-L-glutamic acid dimethyl ester p-toluenesulfonate salt of Formula III; and

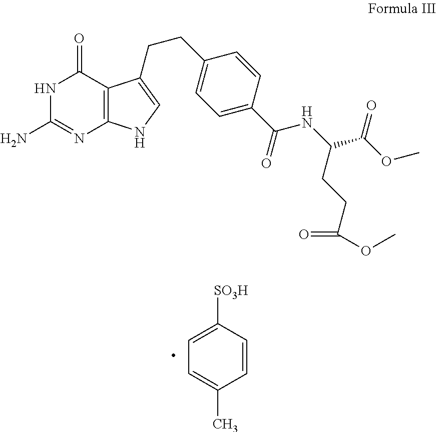

iii) converting the compound of Formula III to pemetrexed disodium using aqueous sodium hydroxide solution at a temperature below 20° C.

In an aspect, the present application provides processes for the preparation of pemetrexed disodium, substantially free from its chiral impurity of Formula C, embodiments comprising reacting N-[4-[2-(2-amino-4,7-dihydro-4-oxo-1H-pyrrolo[2,3-d]pyrimidin-5-yl)ethyl]benzoyl]-L-glutamic acid dimethyl ester p-toluene sulfonate salt of formula III with aqueous sodium hydroxide solution, at temperatures below 20° C.

In an aspect, the present application provides pemetrexed disodium, substantially free from impurities of Formulas A, B, and C.

### BRIEF DESCRIPTION OF THE DRAWING

FIG. 1 is an example of a chromatogram showing the analyses of enantiomers of N-[4-[2-(2-amino-4,7-dihydro-4-oxo-1H-pyrrolo[2,3-d]pyrimidin-5-yl)ethyl]benzoyl]-glutamic acid disodium salt.

### DETAILED DESCRIPTION

In an aspect, the present application provides processes for preparing N-[4-[2-(2-amino-4,7-dihydro-4-oxo-1H-pyrrolo[2,3-d]pyrimidin-5-yl)ethyl]benzoyl]-L-glutamic acid dialkyl ester p-toluenesulfonate salt of formula,

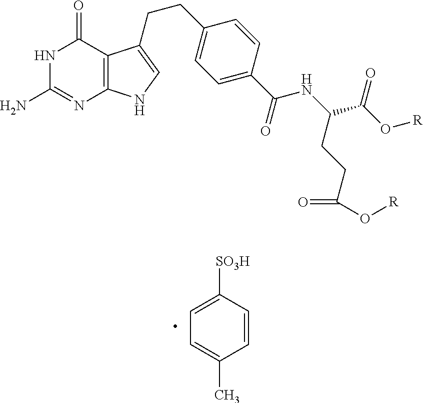

wherein R=alkyl
comprising:

a) reacting 4-[2-(2-amino-4,7-dihydro-4-oxo-1H-pyrrolo[2,3-d]pyrimidin-5-yl)ethyl] benzoic acid of Formula II

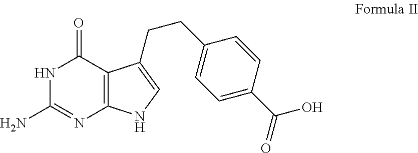 [SMILES: Nc1nc2[nH]cc(CCc3ccc(C(=O)O)cc3)c2c(=O)[nH]1]

with L-dialkyl glutamate HCl, in the presence of N-methylpyrrolidone (NMP), to obtain N-[4-[2-(2-amino-4,7-dihydro-4-oxo-1H-pyrrolo[2,3-d]pyrimidin-5-yl)ethyl]benzoyl]-L-glutamic acid dialkyl ester; and

b) reacting N-[4-[2-(2-amino-4,7-dihydro-4-oxo-1H-pyrrolo[2,3-d]pyrimidin-5-yl)ethyl]benzoyl]-L-glutamic acid dialkyl ester with p-toluenesulfonic acid, in an organic solvent.

In an specific aspect, the present application provides process for preparing N-[4-[2-(2-amino-4,7-dihydro-4-oxo-1H-pyrrolo[2,3-d]pyrimidin-5-yl)ethyl]benzoyl]-L-glutamic acid dimethyl ester p-toluenesulfonate salt of Formula III,

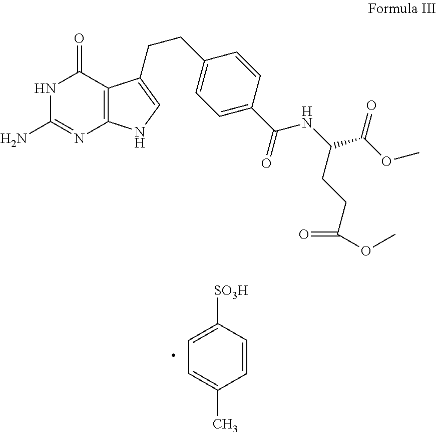

substantially free from its impurity of Formula A, process comprising the steps of:

a) reacting 4-[2-(2-amino-4,7-dihydro-4-oxo-1H-pyrrolo[2,3-d]pyrimidin-5-yl)ethyl] benzoic acid of Formula II

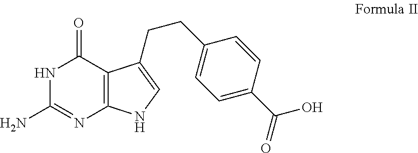 [SMILES: Nc1nc2[nH]cc(CCc3ccc(C(=O)O)cc3)c2c(=O)[nH]1]

with L-dimethyl glutamate HCl, in the presence of N-methylpyrrolidone to obtain a N-[4-[2-(2-amino-4,7-dihydro-4-oxo-1H-pyrrolo[2,3-d]pyrimidin-5-yl)ethyl]benzoyl]L-glutamic acid dimethyl ester; and

b) reacting N-[4-[2-(2-amino-4,7-dihydro-4-oxo-1H-pyrrolo[2,3-d]pyrimidin-5-yl)ethyl]benzoyl]-L-glutamic acid dimethyl ester with p-toluenesulfonic acid in an organic solvent.

Step a) involves reacting 4-[2-(2-amino-4,7-dihydro-4-oxo-1H-pyrrolo[2,3-d]pyrimidin-5-yl)ethyl] benzoic acid of Formula II with L-dimethyl glutamate HCl, in the presence of N-methylpyrrolidone to obtain a N-[4-[2-(2-amino-4,7-dihydro-4-oxo-1H-pyrrolo[2,3-d]pyrimidin-5-yl)ethyl]benzoyl]-L-glutamic acid dimethyl ester.

The reaction is performed in the presence of a coupling agent such as 2-chloro-4,6-dimethoxy-1,3,5-triazine (CDMT), isobutyl chloroformate (IBCf), 1-ethyl-3-(3-dimethylaminopropyl)carbodiimide (EDC), or EDC hydrochloride and HOBt, dicyclohexylcarbodiimide (DCC) and 1-hydroxybenzotriazole (HOBt), optionally in the presence of a base.

In embodiments, the reaction is performed using coupling agents such as 2-chloro-4,6-dimethoxy-1,3,5-triazine (CDMT) or isobutyl chloroformate (IBCf) in the presence of a base such as N-methylmorpholine (NMM), triethylamine, and the like.

In a specific embodiment, the above reaction is performed using 2-chloro-4,6-dimethoxy-1,3,5-triazine (CDMT) in the presence of N-methylmorpholine (NMM).

The condensation reaction is performed at suitable temperatures of about 0° C. to 50° C. The condensation reaction may be performed at any temperatures for any periods of time, to achieve a desired yield and purity. Preferably, the reaction is performed at 30-40° C. to minimize the formation of process related impurities like the compound of Formula G and Formula J.

The inventors of the present application have found that the use of N-methylpyrrolidone (NMP) as the solvent in the above reaction results in an avoidance of formation of the impurity of Formula A in the N-[4-[2-(2-amino-4,7-dihydro-4-oxo-1H-pyrrolo[2,3-d]pyrimidin-5-yl)ethyl]benzoyl]-L-glutamic acid dimethyl ester p-toluenesulfonate salt of Formula III, and subsequently avoidance of formation of the impurity of Formula B or its salt in pemetrexed disodium.

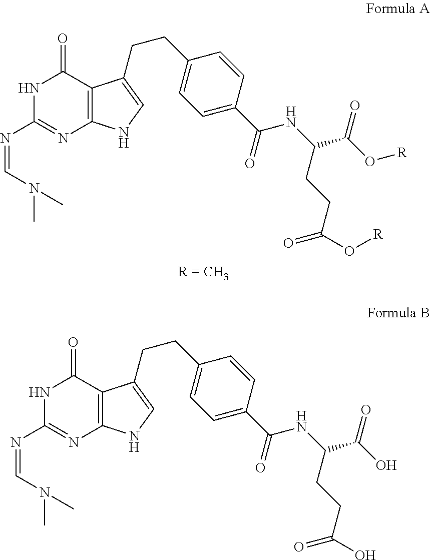

Further, the condensation reaction in the presence of N-methylpyrrolidone (NMP) is cost-effective and enhances industrial applicability.

Advantages for the use of N-methylpyrrolidone (NMP) in the condensation step a) of the above process include:

i) providing a compound of Formula III substantially free from the process-related impurity of Formula A, avoids the formation of the impurity of Formula B in the pemetrexed disodium;

ii) providing substantially complete conversion of the intermediate of Formula II to the compound of Formula III, to remove or reduce the presence of compound of formula II in the pemetrexed disodium as an impurity; and

iii) N-methylpyrrolidone (NMP) is very easy to handle and is not a hazardous chemical.

Step b) involves reacting N-[4-[2-(2-amino-4,7-dihydro-4-oxo-1H-pyrrolo[2,3-d]pyrimidin-5-yl)ethyl]benzoyl]-L-glutamic acid dimethyl ester with p-toluenesulfonic acid in an organic solvent.

N-[4-[2-(2-amino-4,7-dihydro-4-oxo-1H-pyrrolo[2,3-d]pyrimidin-5-yl)ethyl]benzoyl]-L-glutamic acid dimethyl ester obtained in step a) is reacted with p-toluenesulfonic acid to provide the corresponding p-toluene sulfonate salt of Formula III.

In an embodiment, the product obtained from step a) is reacted in situ (i.e., without isolation from the reaction medium) with p-toluenesulfonic acid at a temperature up to boiling point of the solvent to provide the corresponding p-toluenesulfonate salt of Formula III.

The organic solvents useful in step b) include alcohols such as methanol, ethanol, isopropyl alcohol, and the like.

In another aspect, the present application provides process for the preparation of a compound of Formula III, embodiments comprising:

a) reacting 4-[2-(2-amino-4,7-dihydro-4-oxo-1H-pyrrolo[2,3-d]pyrimidin-5-yl)ethyl]benzoic acid of Formula II with L-glutamic acid dimethyl ester hydrochloride salt and 2-chloro-4,6-dimethoxy-1,3,5-triazine (CDMT) and N-methylmorpholine (NMM), in the presence of N-methylpyrrolidone (NMP);

b) adding water and an organic solvent, and extracting the product into the organic solvent;

c) reacting with PTSA in an alcohol to obtain the compound of formula III.

Step a) involves reacting 4-[2-(2-amino-4,7-dihydro-4-oxo-1H-pyrrolo[2,3-d]pyrimidin-5-yl)ethyl]benzoic acid of Formula II with CDMT and NMM and L-glutamic acid dimethyl ester hydrochloride salt, in the presence of N-methylpyrrolidone (NMP).

The reaction is performed at suitable temperatures, such as about 25° C. to 40° C., for any periods of time to achieve the desired compound.

The amount of CDMT used for the condensation reaction is about 0.9 to about 2 molar equivalents, per molar equivalent of the compound of Formula II.

The quantity of N-methylmorpholine used for the condensation reaction may range from about 1 to about 5 molar equivalents, per molar equivalent of the compound of Formula II.

The quantity of L-glutamic acid dimethyl ester hydrochloride salt may range from about 1 to about 1.5 molar equivalents, per molar equivalent of the compound of Formula II.

The quantity of solvent is not critical. However, it is usually minimized to avoid losses of product. In embodiments, the quantity of solvent may range from about 5 to about 15 mL, per gram of the compound of Formula II.

Step b) involves adding water and an organic solvent, and extracting the product into the organic solvent;

After the completion of the reaction water and organic solvent are added to the reaction mixture.

Organic solvents that can be used in step b) include, but are not limited to: halogenated hydrocarbons such as dichloromethane, dichloroethane, chloroform, and carbon tetrachloride; and esters such as ethyl acetate, methyl acetate, and the like.

The obtained reaction product is extracted into the organic solvent at temperatures about 25-35° C. by stirring the reaction mixture for suitable time period and separated from the aqueous layer. The organic layer may be washed with sodium bicarbonate solution. The resultant organic solution may be used directly in the next step or concentrated completely to provide a residue.

Step c) involves a reaction with p-toluenesulfonic acid in an alcohol

The alcohol solvent used in step c) may be selected from methanol, ethanol, isopropyl alcohol, and the like.

The product obtained from step b) is combined with p-toluenesulfonic acid and the alcohol solvent at room temperature and then the mixture is heated to higher temperatures.

In embodiments, the reaction mixture from step b) is combined with p-toluenesulfonic acid and methanol at room temperature and the mixture is heated to higher temperatures, such as from about 50° C. to about 70° C., and maintained for a sufficient period of time, for example, 1-2 hours or longer, and then the mixture may be cooled to lower temperatures to increase the precipitation of a solid.

The solid may be isolated from the reaction suspension using techniques such as filtration by gravity or suction, centrifugation, decantation, and the like.

Optionally, the obtained solid is dried for any desired periods of time, for example, 1 to 10 hours or longer, at any desired temperatures, for example, about 35° C. to about 50° C.

The compound of Formula III obtained from the process of the present application is substantially free of the impurity of Formula A and has a purity greater than about 98%, preferably greater than 99.0% as determined using high performance liquid chromatography (HPLC).

In embodiments, the compound of Formula III contains less than about 0.1% of the impurity of Formula A, by HPLC.

The compound of Formula III prepared as described may be further purified using a suitable technique in the presence of suitable solvent. Suitable techniques include crystallization, recrystallization, solvent anti-solvent techniques, and the like. Suitable solvents include, but are not limited to, C₁C₄ alcohols, N-methylpyrrolidone (NMP), dimethylsulfoxide (DMSO), and any combinations thereof.

An example of the overall process is summarized in the following Scheme 6.

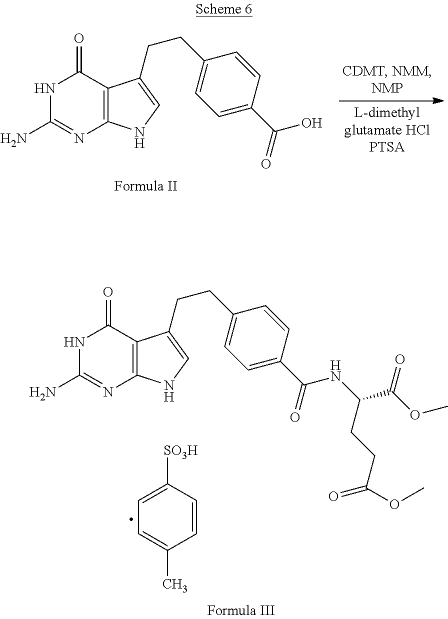 [SMILES: Nc1nc2[nH]cc(CCc3ccc(C(=O)O)cc3)c2c(=O)[nH]1 ; COC(=O)CC[C@H](NC(=O)c1ccc(CCc2c[nH]c3nc(N)[nH]c(=O)c23)cc1)C(=O)OC ; Cc1ccc(S(=O)(=O)O)cc1]

In an aspect, the present application provides processes for the preparation of pemetrexed disodium, substantially free from impurities of Formulas A, B, and C, embodiments comprising:

i) reacting 4-[2-(2-amino-4,7-dihydro-4-oxo-1H-pyrrolo[2,3-d]pyrimidin-5-yl)ethyl]benzoic acid of Formula II:

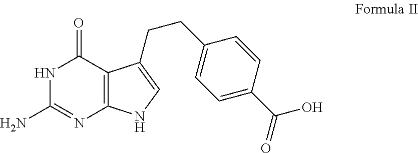 [SMILES: Nc1nc2[nH]cc(CCc3ccc(C(=O)O)cc3)c2c(=O)[nH]1]

with L-dimethyl glutamate hydrochloride, in the presence of N-methylpyrrolidone, to obtain N-[4-[2-(2-amino-4,7-dihydro-4-oxo-1H-pyrrolo[2,3-d]pyrimidin-5-yl)ethyl]benzoyl]-L-glutamic acid dimethyl ester;

ii) reacting N-[4-[2-(2-amino-4,7-dihydro-4-oxo-1H-pyrrolo[2,3-d]pyrimidin-5-yl)ethyl]benzoyl]-L-glutamic acid dimethyl ester with p-toluenesulfonic acid in an organic solvent, to provide N-[4-[2-(2-amino-4,7-dihydro-4-oxo-1H-pyrrolo[2,3-d]pyrimidin-5-yl)ethyl]benzoyl]-L-glutamic acid dimethyl ester p-toluenesulfonate salt of Formula III; and

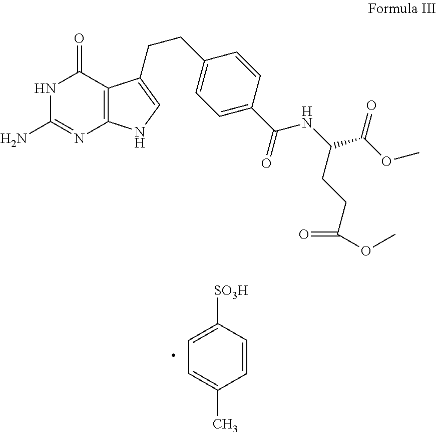

iii) converting the compound of Formula III to pemetrexed disodium using aqueous sodium hydroxide solution at temperatures below about 20° C.

Step i) includes reacting 4-[2-(2-amino-4,7-dihydro-4-oxo-1H-pyrrolo[2,3-d]pyrimidin-5-yl)ethyl]benzoic acid of Formula II with L-dimethyl glutamate HCl, in the presence of N-methylpyrrolidone to obtain N-[4-[2-(2-amino-4,7-dihydro-4-oxo-1H-pyrrolo[2,3-d]pyrimidin-5-yl)ethyl]benzoyl]-L-glutamic acid dimethyl ester.

The condensation reaction may be carried out in the presence of a coupling agent such as CDMT, in the presence of N-methylmorpholine and N-methylpyrrolidone (NMP), to prepare N-[4-[2-(2-amino-4,7-dihydro-4-oxo-1H-pyrrolo[2,3-d]pyrimidin-5-yl)ethyl]benzoyl]-L-glutamic acid dimethyl ester.

Step ii) involves reacting N-[4-[2-(2-amino-4,7-dihydro-4-oxo-1H-pyrrolo[2,3-d]pyrimidin-5-yl)ethyl]benzoyl]-L-glutamic acid dimethyl ester with p-toluenesulfonic acid, in an organic solvent, to provide a compound of Formula III.

The organic solvent used in step ii) can be an alcohol such as methanol, ethanol, isopropyl alcohol, and the like.

Step iii) involves converting N-[4-[2-(2-amino-4,7-dihydro-4-oxo-1H-pyrrolo[2,3-d]pyrimidin-5-yl)ethyl]benzoyl]-L-glutamic acid dimethyl ester p-toluenesulfonate salt of Formula III to pemetrexed disodium, using aqueous sodium hydroxide solution at temperatures below about 20° C.

The reaction is performed at temperatures below 20° C. to avoid the formation of the chiral impurity of Formula C. For example, the temperatures may range from about 0° C. to about 20° C., or about 0° C. to about 5° C.

The present inventors have found that formation of the chiral impurity of Formula C increases when the reaction is performed at higher temperatures, for example, above about 25° C., so lower temperatures generally are used.

The reaction may be performed in the presence of a suitable solvent, such as, but not limited to, water and water-miscible solvents. Water-miscible solvents include: ketones such as acetone, methyl ethyl ketone, and the like; and alcohols such as methanol, ethanol, isopropyl alcohol, and the like.

After completion of the reaction, the reaction mixture is subjected to pH adjustment to values of 7.0-8.5 using an acid, followed by formation of a solid using a suitable organic solvent. Useful acids include hydrochloric acid, hydrobromic acid, acetic acid, and the like. Suitable organic solvents include ketones, alcohols, tetrahydrofuran, acetonitrile, and the like.

An example of the overall process is summarized in the following Scheme 7.

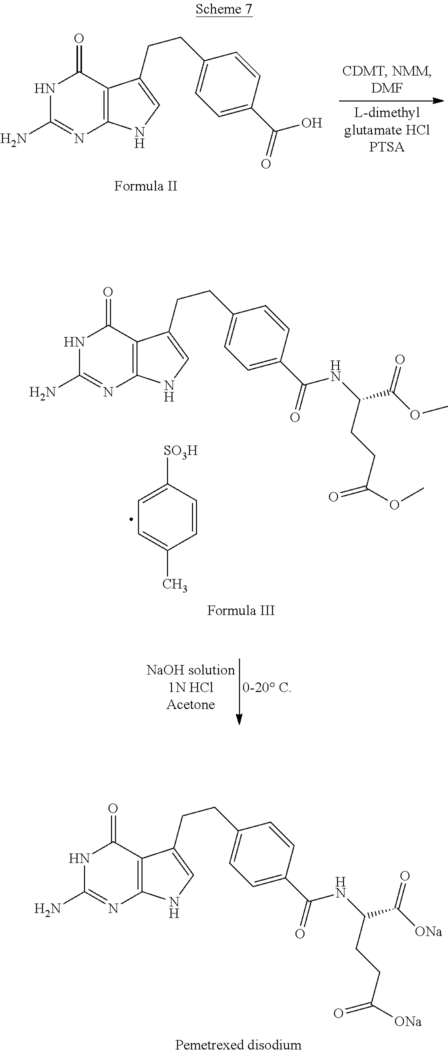

In an aspect, the present application provides processes for the preparation of pemetrexed disodium, substantially free from its chiral impurity of Formula C, embodiments comprising reacting N-[4-[2-(2-amino-4,7-dihydro-4-oxo-1H-pyrrolo[2,3-d]pyrimidin-5-yl)ethyl]benzoyl]-L-glutamic acid dimethyl ester p-toluenesulfonate salt with aqueous sodium hydroxide solution, at temperatures below about 20° C.

In embodiments, the content of a chiral impurity of Formula C in pemetrexed disodium obtained by the process of the present application is less than about 0.1% by weight, as determined using chiral HPLC.

When the reaction is performed between a diester of pemetrexed or its salt, and sodium hydroxide at an ambient temperature or at a higher temperature, for example, higher than about 30° C., the formation of an impurity of Formula C is increased. The inventors of the present application have performed reactions at 25-30° C. and 30-35° C., and found the formation of impurity of Formula C was about 0.15 to 0.2% by weight, and higher, of the impurity at about 1.4 RRT (Pemetrexed=1) as determined using chiral HPLC.

Pemetrexed disodium obtained from N-[4-[2-(2-amino-4,7-dihydro-4-oxo-1H-pyrrolo[2,3-d]pyrimidin-5-yl)ethyl]benzoyl]-L-glutamic acid dimethyl ester p-toluenesulfonate salt at temperatures below 20° C. is substantially free from the chiral impurity of Formula C.

Pemetrexed disodium obtained by the processes of the present application is substantially free from impurities of Formulas A, B, and C, and it may have purity greater than about 99% by weight, preferably greater than about 99.95% by weight as determined using HPLC.

Further, pemetrexed disodium prepared according to the present application is substantially free from impurities of Formula A and Formula B.

In embodiments, the present application provides pemetrexed disodium having less than about 0.1% by HPLC of the impurity of Formula A.

In embodiments, the present application provides pemetrexed disodium having less than about 0.1% by HPLC of the impurity of Formula B.

In embodiments, the present application provides pemetrexed disodium, substantially free from impurities of Formulas A, B, and C.

The term “substantially free” in the present application means the content of the individual impurities in pemetrexed disodium or its intermediate of Formula III is less than about 0.15% by weight.

In embodiments, the content of each independent impurity, or the total drug-related impurities, is less than or equal to about 0.1%, by weight.

A high performance liquid chromatography method for analyzing pemetrexed disodium, used for the examples, is described below.

High performance liquid chromatography is conducted with a BDS HYPERSIL C18 (150×4.6 mm, 3 μm) column and an ultraviolet detector wavelength of 230 nm. The flow rate is 1.0 mL/minute. The mobile phase is comprised of two eluents (A and B). Eluent A is buffer (dissolve 1.36 g of potassium dihydrogen phosphate in 1000 mL of milli-Q-water and adjust the pH of the solution to 3.4±0.05 using dilute phosphoric acid, then filter the solution through a 0.45 μm porosity membrane filter). Eluent B is filtered acetonitrile. Samples of pemetrexed disodium are dissolved in Eluent A and pH of the solution is adjusted to 7±0.05 with dilute potassium hydroxide. The injection volume of sample is about 10 μL and the column temperature is 30±2° C. The samples are carried through the column by gradient elution under the following conditions:

| | Eluent A | Eluent B |
|---|---|---|
| Minutes | (% v/v) | (% v/v) |
| 0 | 95 | 5 |
| 20 | 80 | 20 |
| 30 | 50 | 50 |
| 35 | 80 | 20 |
| 40 | 95 | 5 |
| 45 | 95 | 5 |

The process impurity of Formula B obtained by a prior process, which involves the use of DMF as a solvent, is found in pemetrexed disodium at about 1.23 RRT (Pemetrexed=1).

The relative retention times (RRT) of pemetrexed disodium-related substances are given below (Pemetrexed disodium=1):

| Component | RRT |
|---|---|
| 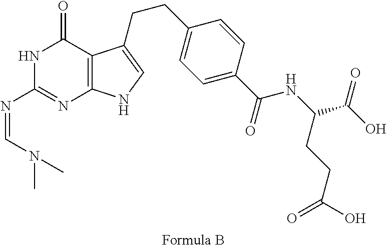 | ~1.23 |
| 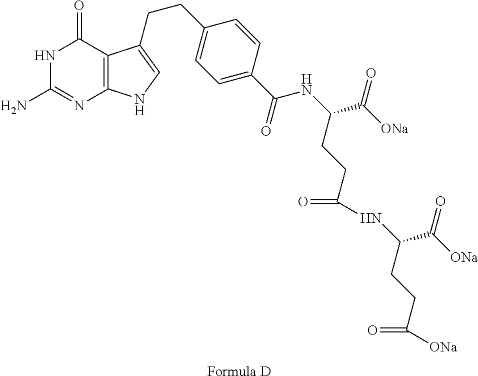 | ~0.89 |
| 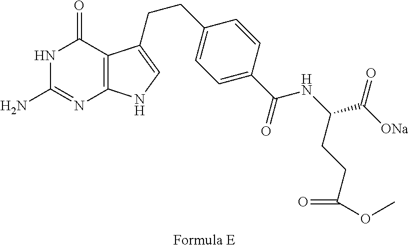 | ~1.37 |
| 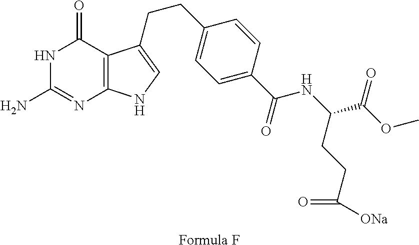 | ~1.40 |

In an embodiment, the present application provides pemetrexed disodium having the content of each of the impurities of the Formula D, Formula E, Formula F, and Formula B less than 0.1% by HPLC.

A chiral HPLC method for analyzing pemetrexed disodium, as used for the examples, includes a CHIRALPAK AD-H (250×4.6 mm, 5 μm) column and a UV detection wavelength of 230 nm. The flow rate is 1.0 mL/minute. Diluent is mobile phase, which is prepared from 5% water in ethanol. The sample injection volume is about 10 μL and the column temperature is 35±2° C.

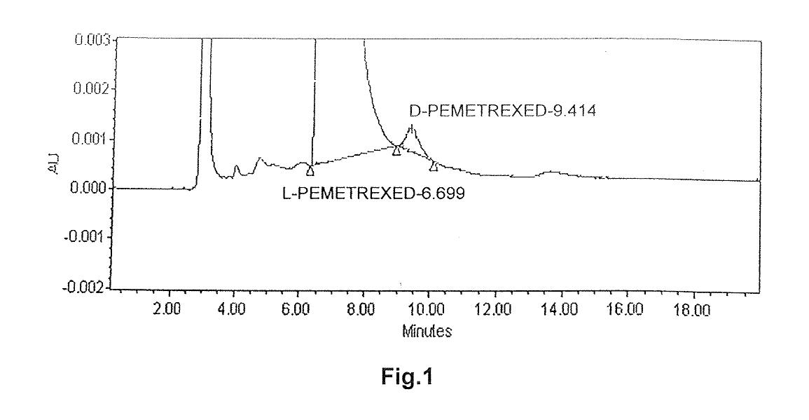

An example of a chromatogram is shown in FIG. 1. Retention time of L-pemetrexed is about 6.8 minutes. The relative retention time (RRT) of D-pemetrexed (chiral impurity of Formula C) is about 1.4 (Pemetrexed=1).

An HPLC method for analyzing the compound of Formula III for the examples below uses a BDS HYPERSIL C18 (150×4.6 mm, 3 μm) column and a UV detection wavelength of 230 nm. The flow rate is 1.0 mL/minute. The mobile phase is comprised of two eluents (A and B). Eluent A is buffer (dissolve 1.36 g of potassium dihydrogen phosphate in 1000 mL of milli-Q-water, adjust the pH of the solution to 3.4±0.05 using dilute phosphoric acid, and filter the solution through a 0.45 μm porosity membrane filter). Eluent B is filtered acetonitrile. Samples of pemetrexed disodium are dissolved in a mixture of methanol and Eluent A (1:4 by volume). The injection volume of sample is about 10 μL and the column temperature is 30±2° C. The samples are carried through the column by gradient elution under the following conditions:

| | Eluent A | Eluent B |
|---|---|---|
| Minutes | (% v/v) | (% v/v) |
| 0 | 90 | 10 |
| 30 | 80 | 20 |
| 40 | 30 | 70 |
| 50 | 30 | 70 |
| 55 | 90 | 10 |
| 60 | 90 | 10 |

The relative retention times (RRT) of the compound of Formula III-related substances are given below (Formula III=1):

| Component | RRT |
|---|---|
| 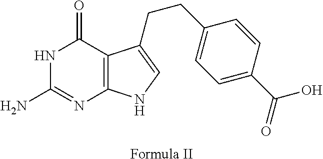 [SMILES: Nc1nc2[nH]cc(CCc3ccc(C(=O)O)cc3)c2c(=O)[nH]1 ; "Formula II"] | ~0.59 |
| 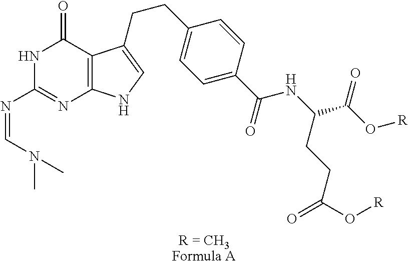 | ~1.06 |
| 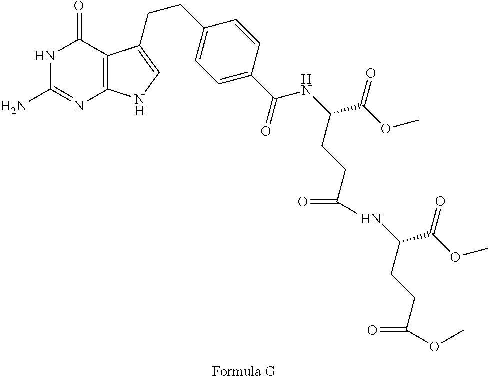 [SMILES: COC(=O)CC[C@H](NC(=O)CC[C@H](NC(=O)c1ccc(CCc2c[nH]c3nc(N)[nH]c(=O)c23)cc1)C(=O)OC)C(=O)OC ; "Formula G"] | ~1.03 |
| 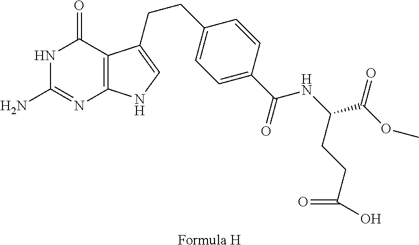 [SMILES: COC(=O)[C@H](CCC(=O)O)NC(=O)c1ccc(CCc2c[nH]c3nc(N)[nH]c(=O)c23)cc1 ; "Formula H"] | ~0.65 |
| 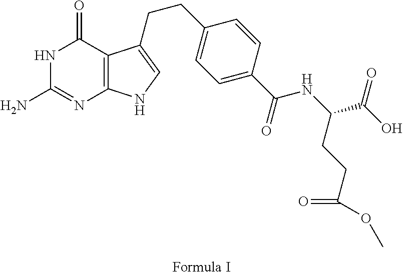 [SMILES: COC(=O)CC[C@H](NC(=O)c1ccc(CCc2c[nH]c3nc(N)[nH]c(=O)c23)cc1)C(=O)O ; "Formula I"] | ~0.62 |
| 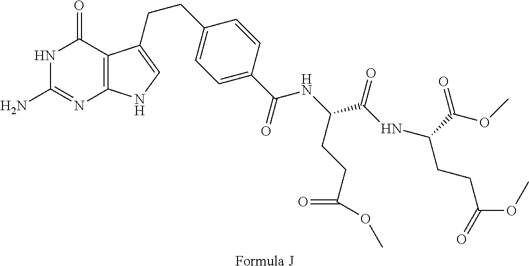 [SMILES: COC(=O)CC[C@H](NC(=O)c1ccc(CCc2c[nH]c3nc(N)[nH]c(=O)c23)cc1)C(=O)N[C@@H](CCC(=O)OC)C(=O)OC ; "Formula J"] | ~1.05 |

In an embodiment, the present application provides a compound of formula III substantially free of the impurities listed in the above table.

In an embodiment, the present application provides a compound of formula III having the content of each of the impurities of the Formula A, Formula G, and Formula J less than 0.15% by HPLC.

The present application includes pharmaceutical compositions comprising pemetrexed disodium, substantially free from impurities of Formulas A, B, and C, together with at least one pharmaceutically acceptable excipient.

The present application includes pharmaceutical compositions comprising pemetrexed disodium, substantially free from the impurity of Formula B, together with at least one pharmaceutically acceptable excipient.

The present application also includes pharmaceutical compositions comprising pemetrexed disodium, substantially free of the chiral impurity of Formula C, together with at least one pharmaceutically acceptable excipient.

As is known in the art, suitable pharmaceutical compositions may be formulated as: liquid compositions for oral administration including, for example, solutions, suspensions, syrups, elixirs, and emulsions; compositions for parenteral administration, such as, suspensions, emulsions, or aqueous or non-aqueous sterile solutions; and solid oral dosage forms, such as filled hard gelatin capsules, compressed tablets, and gel caps, wherein the pemetrexed disodium is suspended, dissolved, dispersed, or emulsified in a vehicle surrounded by a soft capsule material.

Pharmaceutically acceptable excipients that are of use in the present application include, but are not limited to: diluents such as starches, pregelatinized starch, lactose, powdered cellulose, microcrystalline cellulose, dicalcium phosphate, tricalcium phosphate, mannitol, sorbitol, sugar, and the like; binders, such as acacia, guar gum, tragacanth, gelatin, polyvinylpyrrolidones, hydroxypropyl celluloses, hydroxypropyl methylcelluloses, pregelatinized starches, and the like; disintegrants, such as starch, sodium starch glycolate, pregelatinized starch, crospovidones, croscarmellose sodium, colloidal silicon dioxide, and the like; lubricants, such as stearic acid, magnesium stearate, zinc stearate, and the like; glidants, such as colloidal silicon dioxide and the like; solubility or wetting enhancers, such as anionic, cationic, and neutral surfactants, complex forming agents, such as various grades of cyclodextrins; and release rate controlling agents, such as hydroxypropyl celluloses, hydroxymethyl celluloses, hydroxypropyl methylcelluloses, ethyl celluloses, methyl celluloses, various grades of methyl methacrylates, waxes, and the like. Other pharmaceutically acceptable excipients that are of use include, but are not limited to, any one or more of film formers, plasticizers, colorants, flavoring agents, sweeteners, viscosity enhancers, preservatives, antioxidants, and the like.

Certain specific aspects and embodiments will be described in greater detail with reference to the following examples, which are provided only for purposes of illustration and should not be construed as limiting the scope of this application in any manner.

### REFERENCE EXAMPLE

### Preparation of N-[4-[2-(2-amino-4,7-dihydro-4-oxo-1H-pyrrolo[2,3-d]pyrimidin-5-yl)ethyl]benzoyl]-L-glutamic acid dimethyl ester p-toluene sulfonate salt (using dimethylformamide solvent)

Dimethylformamide (210 mL), 4-[2-(2-amino-4,7-dihydro-4-oxo-1H-pyrrolo[2,3-d]pyrimidin-5-yl)ethyl]benzoic acid (30 g) and N-methylmorpholine (31.2 mL) are combined in a flask with stirring at room temperature. 2-Chloro-4,6-dimethoxy-1,3,5-triazine (CDMT; 19.4 g) is added under a nitrogen atmosphere and the temperature is raised to 30-35° C. and maintained for 1-2 hours. L-glutamic acid dimethyl ester hydrochloride salt (23.2 g) is added under a nitrogen atmosphere and the mixture is stirred for 1 hour. Water (300 mL) and dichloromethane (300 mL) are added, the mixture is stirred for 15 minutes, and the layers are separated. The aqueous layer is extracted with dichloromethane (150 mL). The dichloromethane layers are combined and washed with 7% sodium bicarbonate solution (150 mL), and then concentrated completely to get an oily residue. Methanol (1.2 L) and p-toluenesulfonic acid (28.5 g) are combined with the residue at room temperature, then the temperature is raised to 60-65° C. and maintained for 2-3 hours. The suspension is cooled to 25-30° C. and filtered. The solid is washed with methanol (90 mL), suction dried, and then dried at 45° C. for 2-3 hours, to afford 15 g of the title compound.

Purity: 98.9% by HPLC.

Impurity of Formula A: 0.29% at about 1.06 RRT.

### Example 1

### Preparation of N-[4-[2-(2-amino-4,7-dihydro-4-oxo-1H-pyrrolo[2,3-d]pyrimidin-5-yl)ethyl]benzoyl]-L-glutamic acid dimethyl ester p-toluenesulfonate salt (Formula III)

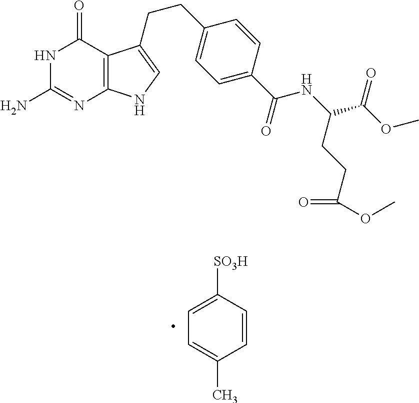

N-Methyl-2-pyrrolidone (10 mL) and 4-[2-(2-amino-4,7-dihydro-4-oxo-1H-pyrrolo[2,3-d]pyrimidin-5-yl)ethyl]benzoic acid (1 g) are charged into a round bottom flask under nitrogen atmosphere and stirred at room temperature. N-methylmorpholine (1.06 mL) and 2-chloro-4,6-dimethoxy-1,3,5-triazine (0.65 g) are added to the reaction mixture, heated to 30-35° C. and maintained for 1 hour. L-glutamic acid dimethyl ester hydrochloride salt (0.78 g) is added and the mixture is stirred for 1 hour. Water (10 mL) and dichloromethane (10 mL) are added, stirred for 10-20 minutes and the dichloromethane layer is separated. The aqueous layer is extracted with dichloromethane (10 mL). The dichloromethane layers are combined, washed with saturated sodium bicarbonate solution (10 mL), and then concentrated completely to obtain an oily residue. The residue is dissolved in methanol (20 mL). p-Toluenesulfonic acid (1.59 g) in methanol (20 mL) is added at room temperature, and the temperature is raised to 60-65° C. and maintained for 1-2 hours. The suspension is cooled to 25-30° C. and filtered. The solid is washed with methanol (10 mL) and dried at 45° C. for 5 hours, to afford 1.08 g of title compound.

Yield: 51%.

Purity: 98.36% at 36.63 RT.

Impurity of Formula A: Not detected.

Unidentified impurities: 0.03% at ˜1.04 RRT; 0.02% at ˜1.09 RRT; 0.11% at ˜1.1 RRT; 0.29% at 0.607 ˜RRT; 0.42% at ˜0.637 RRT.

Impurity 4-[2-(2-amino-4,7-dihydro-4-oxo-1H-pyrrolo[2,3-d]pyrimidin-5-yl)ethyl]benzoic acid methyl ester: 0.17% at ˜1.1 RRT.

### Example 2

### Preparation of Pemetrexed Disodium

N-[4-[2-(2-amino-4,7-dihydro-4-oxo-1H-pyrrolo[2,3-d]pyrimidin-5-yl)ethyl]benzoyl]-L-glutamic acid dimethyl ester p-toluenesulfonate salt (5 g) prepared according to Example 1 is dissolved in sodium hydroxide solution (1.91 g of sodium hydroxide dissolved in 95.6 L of water) at 0-5° C. under nitrogen and stirred for 45 minutes. The pH is adjusted to 7.81 by adding 1N HCl solution (15 mL). The mixture is added to acetone (300 mL) under nitrogen, stirred for 1 hour and filtered. The solid is washed with acetone (15 mL) and dried at 30-35° C. under vacuum for 5 hours, to afford 4.1 g of the title compound.

Purity: 99.69%; Impurity of Formula B: not detected by HPLC.

Chiral purity: 99.96%; chiral impurity of Formula C, 0.04% at ˜1.39 RRT.

### Example 3

### Preparation of the Impurity of Formula A, where R is Methyl

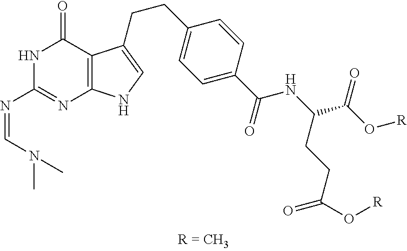

N-[4-[2-(2-amino-4,7-dihydro-4-oxo-1H-pyrrolo[2,3-d]pyrimidin-5-yl)ethyl]benzoyl]-L-glutamic acid dimethyl ester p-toluene sulfonate salt (20 g) is suspended in dimethylformamide dimethylacetal (dimethylformamide dimethyl acetal represents DMF-DMA) (150 mL) at room temperature with stirring. p-Toluenesulfonic acid (130.3 g) is added and the mixture is heated to 60-65° C. and stirred for 2-3 hours at the same temperature. The solution is cooled to room temperature. Dichloromethane (200 mL) and water (200 mL) are added and stirred for 15 minutes. The layers are separated and the aqueous layer is extracted with dichloromethane (200 mL). The combined organic layers are concentrated completely under vacuum at 45-50° C. to obtain a thick liquid mass. Water (500 mL) is added to the thick liquid mass and stirred for 4 hours at room temperature. The suspension is filtered and the solid is washed with diisopropyl ether (100 mL) and dried for 4 hours at 45-50° C. under vacuum. The solid is dissolved in methanol (48 mL) and diisopropyl ether (400 mL) is added slowly over 45-60 minutes, then the mixture is stirred for 1 hour at room temperature. The obtained suspension is filtered and solid is washed with diisopropyl ether (50 mL) and dried for 4 hours at 45-50° C. under vacuum, to afford 12.5 g of the title compound.

Purity: 93.4%.

MASS analysis: M-1 peak: 509.3.

¹H NMR (DMSO-d6, 400 MHz): δ 2.0-2.2 (m, 2H); 2.45 (t, J=7.2, 2H); 2.89-3.04 (m, 4H); 3.01 (s, 3H); 3.11 (s, 3H); 3.58 (s, 3H); 3.64 (s, 3H); 4.46 (m, 1H); 6.47 (s, 1H); 7.31 (d, J=8.0, 2H); 7.78 (d, J=8.0, 2H); 8.48 (s, 1H); 8.69 (d, J=7.6, 1H); 10.74 (br, s); 10.82 (br, s).

### Example 4

### Preparation of the Impurity of Formula B

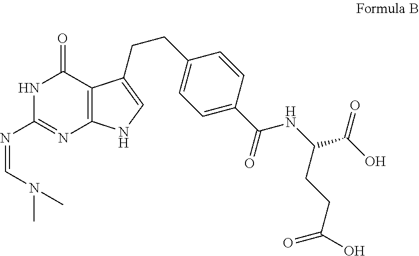

The impurity of Formula A (5 g), prepared according to Example 3, is added to a solution of sodium hydroxide (1.6 g of sodium hydroxide is dissolved in 80 mL of water) at 0-5° C. and stirred for 10 minutes. Ethanol (80 mL) is added to the solution and stirred for 1 hour at 0-5° C. The pH is adjusted to 7-8 with 1N HCl (15 mL). Ethanol (220 mL) is added under nitrogen and stirred for 30 minutes. The solution is concentrated completely at 45-50° C. under vacuum, to obtain a gummy mass. Methanol (100 mL) is added to the gummy mass to obtain a solid, which is collected.

The obtained solid is suspended in methanol (25 mL) at 0-5° C. and stirred for 60 minutes. The suspension is filtered and the solid is washed with methanol (5 mL) and dried at 45-50° C., to obtain 3.0 g of the title compound.

The methanol purification is repeated and the solid is dried for 4 hours at 30-35° C. under vacuum to obtain 2.0 g of purified title compound.

Purity: 93.79% by HPLC at 18.552 RT (1.21 ˜RRT); Pemetrexed disodium: 2.38% at 15.324 RT.

MASS: M-1: 481.3.

¹H NMR (DMSO-d6, 400 MHz): δ 1.95-2.11 (m, 2H); 2.45 (t, J=7.6, 2H); 2.89-3.1 (m, 4H); 3.0 (s, 3H); 3.1 (s, 3H); 4.2 (m, 1H); 6.45 (s, 1H); 7.26 (d, J=7.6, 2H); 7.74 (d, J=7.6, 2H); 8.12 (d, J=7.6, 1H); 8.48 (s, 1H); 10.74 (br, s); 10.82 (br, s).

### Example 5

### Preparation of Pemetrexed Disodium

N-[4-[2-(2-amino-4,7-dihydro-4-oxo-1H-pyrrolo[2,3-d]pyrimidin-5-yl)ethyl]benzoyl]-L-glutamic acid dimethyl ester p-toluene sulfonate salt (5.0 g), prepared according to Reference Example (purity: 98.9% by HPLC), is dissolved in sodium hydroxide solution (1.91 g of sodium hydroxide dissolved in 96 mL of water) at 0-5° C. with stirring under nitrogen bubbling. The reaction solution is stirred for 45 minutes at a same temperature, and filtered. The filtrate pH is adjusted to 7.5-8 by adding 1N HCl solution (17 mL). Acetone (300 mL) is added to the solution at 25-30° C., stirred for 1 hour, and the suspension is filtered. The solid is washed with acetone (15 mL) and dried at 30-35° C. under vacuum for 2 hours, to afford 4.2 g of the title compound. Yield: 88.4%.

Purity: 98.91% by HPLC.

Impurity of Formula B: 0.17% at ˜1.23 RRT

Chiral Impurity of Formula C, 0.04% at ˜1.4 RRT.

### Example 6

### Preparation of Pemetrexed Disodium at 30-35° C.

N-[4-[2-(2-amino-4,7-dihydro-4-oxo-1H-pyrrolo[2,3-d]pyrimidin-5-yl)ethyl]benzoyl]-L-glutamic acid dimethyl ester p-toluene sulfonate salt (100 g), prepared according to (Reference Example) (purity: 98.9% by HPLC), is dissolved in sodium hydroxide solution (28.7 g of sodium hydroxide dissolved in 1.43 L of water) at 30-35° C. with stirring under nitrogen bubbling. The solution is stirred for 45 minutes at the same temperature and filtered. The filtrate pH is adjusted to 7.5-8 by adding 1N HCl solution (172 mL). Ethanol (6 L) is added at 25-30° C., the mixture is stirred for 1 hour and the suspension is filtered. The solid is washed with ethanol (120 mL) and dried at 35° C. under vacuum for 3 hours, to afford 84.6 g of the title compound.

Purity: 99.74%; impurity of Formula B: 0.04% at 1.23 RRT by HPLC.

Chiral purity: 99.86%; Chiral impurity of Formula C 0.14% by chiral HPLC.

### Example 7

### Purification of the Compound of Formula III

N-[4-[2-(2-amino-4,7-dihydro-4-oxo-1H-pyrrolo[2,3-d]pyrimidin-5-yl)ethyl]benzoyl]-L-glutamic acid dimethyl ester p-toluene sulfonate salt (205 g) is dissolved in N-methylpyrrolidone (300 mL) at room temperature. Methanol (6 L) is added and the temperature is raised to 60-65° C. and maintained for 1 hour. The mixture is cooled to room temperature, filtered, and the solid is washed with methanol (3 mL) and dried for 4 hours at 40-45° C. under vacuum, to afford 170 g of purified title compound.

Purity: 99.39% by HPLC.

Impurities: 0.25% at 0.64 RRT; 0.11% at 0.65 RRT; 0.06% at 1.048 RRT; 0.03% at 1.03 RRT.

Volatile impurities: NMP content: 170 ppm by gas chromatography.

Moisture content by Karl Fisher: 0.5%.

1. A process for preparing N-[4-[2-(2-amino-4,7-dihydro-4-oxo-1H-pyrrolo[2,3-d]pyrimidin-5-yl)ethyl]benzoyl]-L-glutamic acid dialkyl ester p-toluenesulfonate salt of the formula 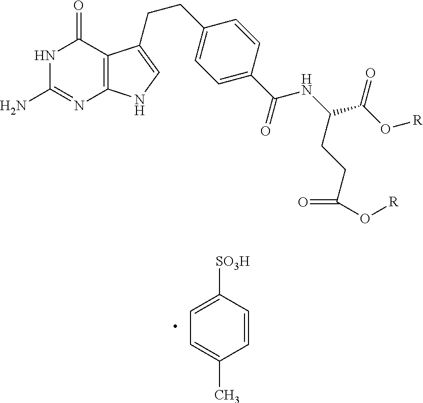
wherein R=alkyl, comprising:
a) reacting 4-[2-(2-amino-4,7-dihydro-4-oxo-1H-pyrrolo[2,3-d]pyrimidin-5-yl)ethyl] benzoic acid of Formula II 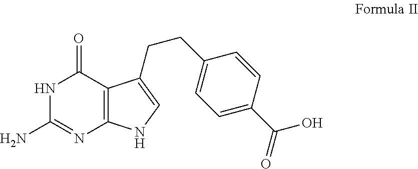 [SMILES: Nc1nc2[nH]cc(CCc3ccc(C(=O)O)cc3)c2c(=O)[nH]1]
with L-dialkyl glutamate HCl, in the presence of N-methylpyrrolidone (NMP), to obtain N-[4-[2-(2-amino-4,7-dihydro-4-oxo-1H-pyrrolo[2,3-d]pyrimidin-5-yl)ethyl]benzoyl]-L-glutamic acid dialkyl ester; and
b) reacting N-[4-[2-(2-amino-4,7-dihydro-4-oxo-1H-pyrrolo[2,3-d]pyrimidin-5-yl)ethyl]benzoyl]-L-glutamic acid dialkyl ester with p-toluenesulfonic acid, in an organic solvent.

2. The process according to claim 1, preparing N-[4-[2-(2-amino-4,7-dihydro-4-oxo-1H-pyrrolo[2,3-d]pyrimidin-5-yl)ethyl]benzoyl]-L-glutamic acid dimethyl ester p-toluenesulfonate salt of Formula III, 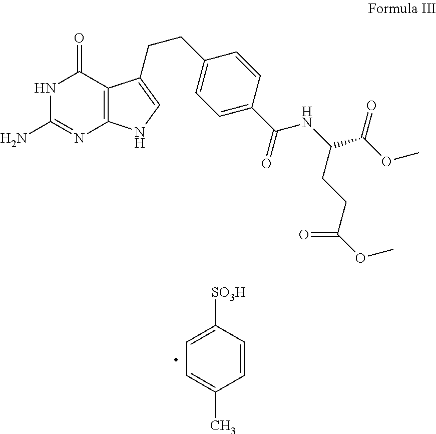
substantially free from an impurity of Formula A, 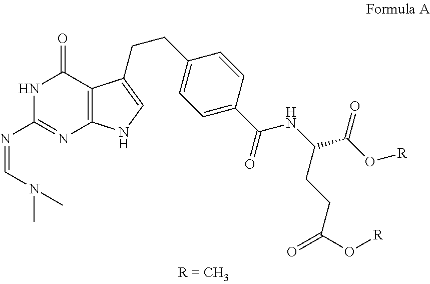
comprising:
a) reacting 4-[2-(2-amino-4,7-dihydro-4-oxo-1H-pyrrolo[2,3-d]pyrimidin-5-yl)ethyl] benzoic acid of Formula II 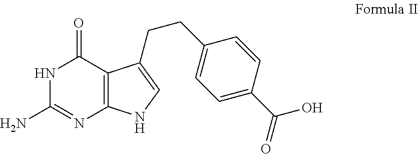 [SMILES: Nc1nc2[nH]cc(CCc3ccc(C(=O)O)cc3)c2c(=O)[nH]1]
with L-dimethyl glutamate HCl, in the presence of N-methylpyrrolidone to obtain N-[4-[2-(2-amino-4,7-dihydro-4-oxo-1H-pyrrolo[2,3-d]pyrimidin-5-yl)ethyl]benzoyl]-L-glutamic acid dimethyl ester; and
b) reacting N-[4-[2-(2-amino-4,7-dihydro-4-oxo-1H-pyrrolo[2,3-d]pyrimidin-5-yl)ethyl]benzoyl]-L-glutamic acid dimethyl ester with p-toluenesulfonic acid in an organic solvent.

3. The process according to claim 1, wherein step a) is carried out in the presence of a coupling agent and a base.

4. The process according to claim 3, wherein a coupling agent is 2-chloro-4,6-dimethoxy-1,3,5-triazine, isobutyl chloroformate, dicyclohexylcarbodiimide and 1-hydroxybenzotriazole, 1-ethyl-3-(3-dimethylaminopropyl)carbodiimide, or its hydrochloride and 1-hydroxybenzotriazole.

5. The process according to claim 3, wherein the base is N-methyl morpholine or triethylamine.

6. The process according to claim 3, wherein a coupling agent is 2-chloro-4,6-dimethoxy-1,3,5-triazine and a base is N-methylmorpholine.

7. The process according to claim 1, wherein the reaction of step a) is conducted at temperatures about 0° C. to about 50° C.

8. The process according to claim 2, comprising:
a) reacting 4-[2-(2-amino-4,7-dihydro-4-oxo-1H-pyrrolo[2,3-d]pyrimidin-5-yl) ethyl]benzoic acid of Formula II with L-glutamic acid dimethyl ester hydrochloride salt and 2-chloro-4,6-dimethoxy-1,3,5-triazine and N-methylmorpholine, in the presence of N-methylpyrrolidone; 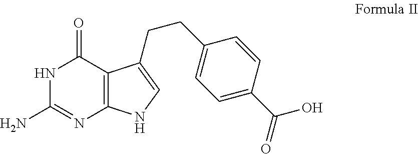 [SMILES: Nc1nc2[nH]cc(CCc3ccc(C(=O)O)cc3)c2c(=O)[nH]1]
b) adding water and an organic solvent, followed by extracting the product into the organic layer; and
c) reacting with p-toluenesulfonic acid in an alcohol, followed by heating the reaction mixture.

9. N-[4-[2-(2-amino-4,7-dihydro-4-oxo-1H-pyrrolo[2,3-d]pyrimidin-5-yl)ethyl]benzoyl]-L-glutamic acid dimethyl ester p-toluenesulfonate salt of Formula III, having less than about 0.1% of an impurity of Formula A.

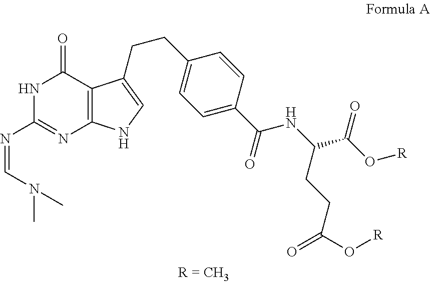

10. A process for preparing pemetrexed disodium substantially free from impurities of Formulas A, B, and C, comprising: 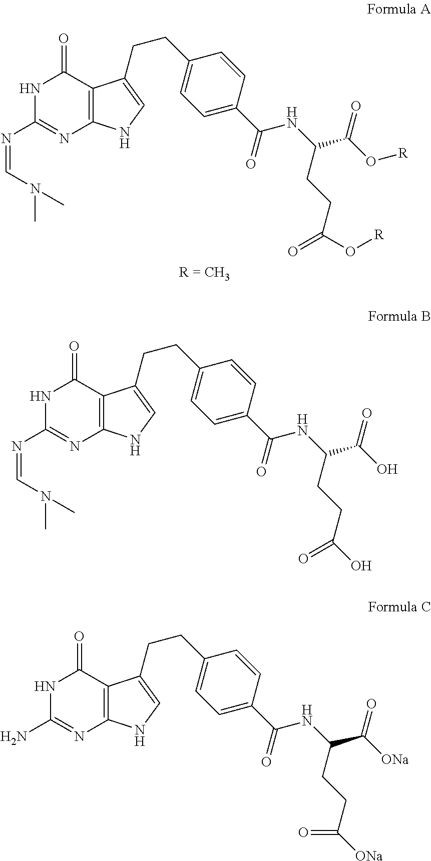
i) reacting 4-[2-(2-amino-4,7-dihydro-4-oxo-1H-pyrrolo[2,3-d]pyrimidin-5-yl)ethyl]benzoic acid of Formula II 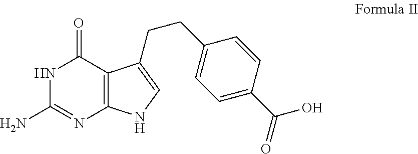 [SMILES: Nc1nc2[nH]cc(CCc3ccc(C(=O)O)cc3)c2c(=O)[nH]1]
with L-dimethyl glutamate hydrochloride, in the presence of N-methylpyrrolidone, to obtain N-[4-[2-(2-amino-4,7-dihydro-4-oxo-1H-pyrrolo[2,3-d]pyrimidin-5-yl)ethyl]benzoyl]-L-glutamic acid dimethyl ester;
ii) reacting N-[4-[2-(2-amino-4,7-dihydro-4-oxo-1H-pyrrolo[2,3-d]pyrimidin-5-yl)ethyl]benzoyl]-L-glutamic acid dimethyl ester with p-toluenesulfonic acid in an organic solvent to provide a N-[4-[2-(2-amino-4,7-dihydro-4-oxo-1H-pyrrolo[2,3-d]pyrimidin-5-yl)ethyl]benzoyl]-L-glutamic acid dimethyl ester p-toluenesulfonate salt of Formula III; and 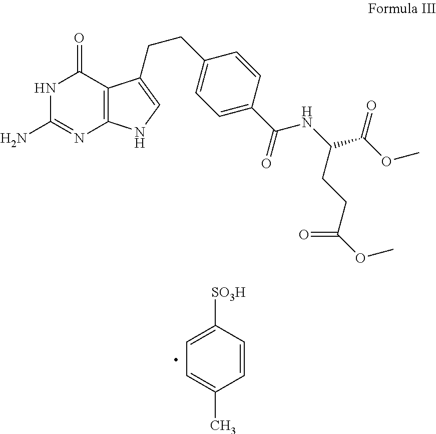
iii) converting the compound of Formula III to pemetrexed disodium using aqueous sodium hydroxide solution at temperatures below about 20° C.

11. A process for the preparation of pemetrexed disodium, substantially free from a chiral impurity of Formula C, 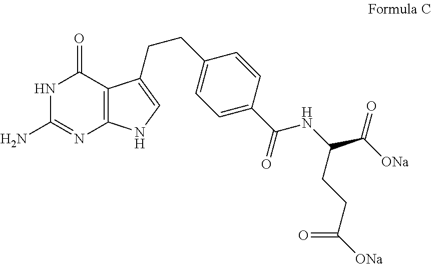
comprising reacting N-[4-[2-(2-amino-4,7-dihydro-4-oxo-1H-pyrrolo[2,3-d]pyrimidin-5-yl)ethyl]benzoyl]-L-glutamic acid dimethyl ester p-toluene sulfonate salt of Formula III with aqueous sodium hydroxide solution, at temperatures below about 20° C.

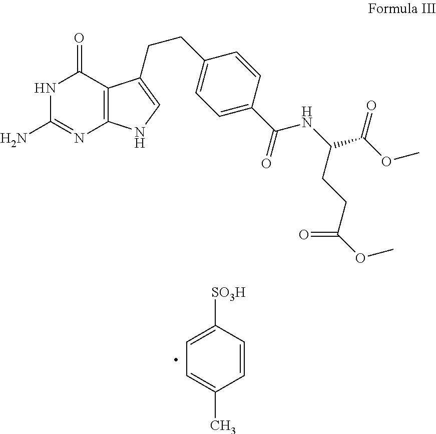

12. Pemetrexed disodium having less than 0.1% by weight of an impurity of Formula A.

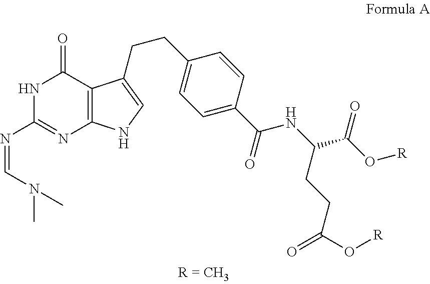

13. Pemetrexed disodium having less than 0.1% by weight of an impurity of Formula B.

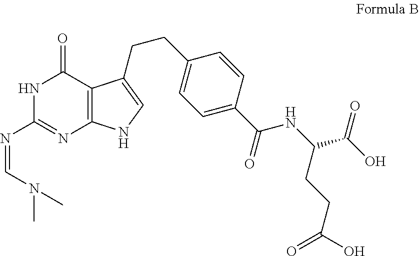

14. Pemetrexed disodium having less than 0.1% by weight of an impurity of Formula C.

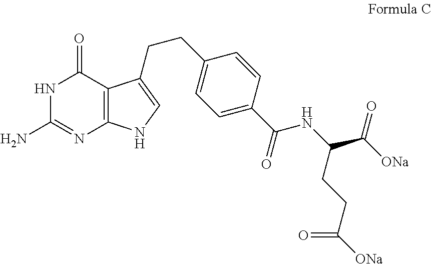

15. Pemetrexed disodium, substantially free from impurities of Formulas A, B, C, D, E, and F.

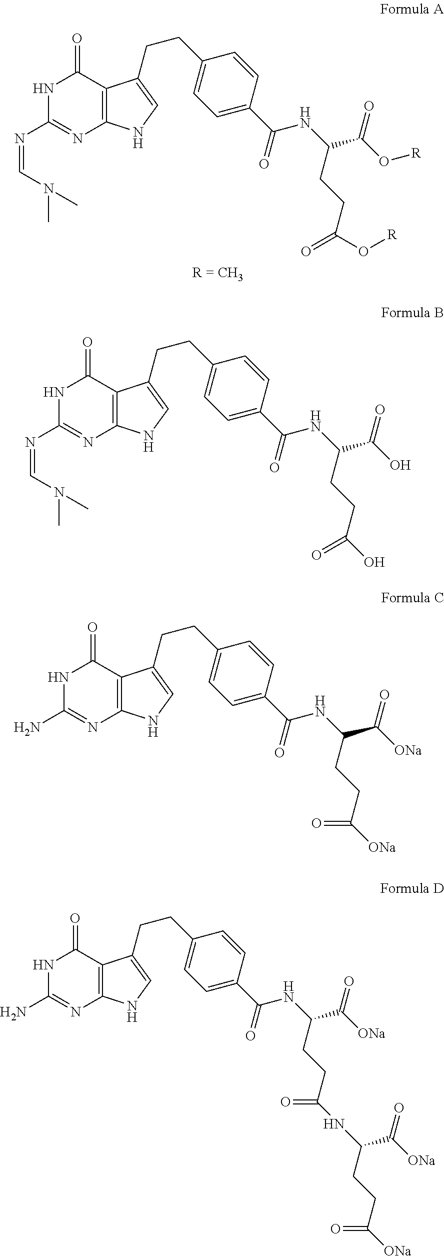

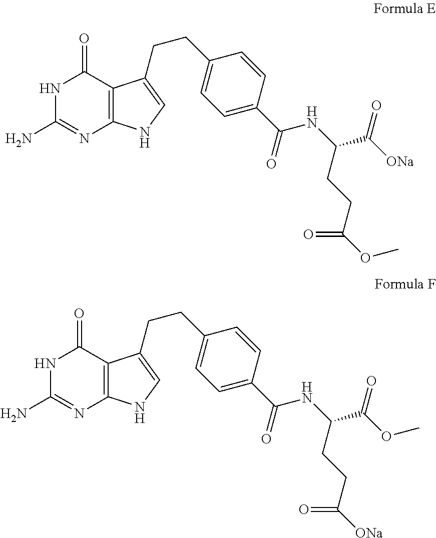

16. (canceled)
